(19) United States
(12) Patent Application Publication
(10) Pub. No.: US 2020/0353009 A1
(43) Pub. Date: Nov. 12, 2020

(54) PHARMACEUTICAL COMPOSITION FOR PREVENTING OR TREATING NEURODEGENERATIVE DISEASE, CONTAINING, AS ACTIVE INGREDIENTS, CYCLODEXTRIN AND STEM CELLS IN WHICH VEGF IS OVEREXPRESSED

(71) Applicant: KYUNGPOOK NATIONAL UNIVERSITY INDUSTRY-ACADEMIC COOPERATION FOUNDATION, Daegu (KR)

(72) Inventors: Jae Sung BAE, Daegu (KR); Hee Kyung JIN, Daegu (KR)

(21) Appl. No.: 16/634,869

(22) PCT Filed: Jul. 3, 2018
(86) PCT No.: PCT/KR2018/007503

§ 371 Date: Jan. 28, 2020

(30) Foreign Application Priority Data
Jul. 28, 2017 (KR) 10-2017-0096511

(51) Int. Cl. A61K 35/30; A61K 31/724; A61K 38/18; A61P 25/28

(52) U.S. Cl. CPC A61K 35/30; A61K 31/724; A61K 9/0019; A61P 25/28; A61K 38/1866

(57) ABSTRACT

The present invention relates to a pharmaceutical composition for preventing or treating neurodegenerative disease, containing, as active ingredients, cyclodextrin and stem cells in which VEGF is overexpressed. A combined treatment of cyclodextrin and stem cells in which VEGF is overexpressed has remarkable synergistic effects, with respect to therapeutic efficacy on the disease, such as a lifespan increase, mobility improvement, inhibition of neurogenic inflammation, inhibition of nerve cell apoptosis and inhibition of lipid accumulation in organs, including the brain, on a neurodegenerative disease model, thereby presenting a novel therapeutic strategy.

### TECHNICAL FIELD

This application claims the priority of Korean Patent Application No. 10-2017-0096511, filed on Jul. 28, 2017, the entirety of which is a reference of the present application.

The present invention relates to a pharmaceutical composition for preventing or treating neurodegenerative diseases containing cyclodextrin and VEGF-overexpressed stem cells as active ingredients.

### BACKGROUND ART

With the rise of older populations worldwide, neurodegenerative diseases (NDDs) are expected to catch up with cancer, the second leading cause of death following cardiovascular diseases. Accordingly, the market of agents for treating neurodegenerative diseases has been growing at a high rate of 20% since 2000. As such, interest in neurodegenerative diseases is increasing day by day.

The neurodegenerative diseases are diseases in which neuronal cells are gradually destroyed to cause loss of cognitive ability and mobility function, leading to death. Niemann-pick disease, Alzheimer's disease (AD), Parkinson's disease (PD), etc. are typical, and the incidence of the diseases increases with age. The neurodegenerative diseases show common characteristics such as neuronal cell death, brain capacity reduction, and nerve inflammation. Neurodegenerative diseases such as Niemann-pick disease, Alzheimer's disease (AD), Parkinson's disease (PD), Huntington's disease, and Lou Gehrig's disease are known to be closely related to changes in cholesterol and lipid metabolism (Caroline Coisne et al., Cyclodextrins as Emerging Therapeutic Tools in the Treatment of Cholesterol-Associated Vascular and Neurodegenerative Diseases, Molecules 2016, 21, 1748; Rao Muralikrishna Adibhatla et al., Role of Lipids in Brain Injury and Diseases, Future Lipidol. 2007 August; 2(4): 403-422.). In addition, it has been reported that lipid accumulation in the brain caused due to disorders of lipid metabolism in Gaucher disease, Fabry disease, Tay-Sachs disease and Sandhoff disease (Nature. 2014 Jun. 5; 510(7503):68-75, Trends Cell Biol. 2003 April; 13(4):195-203., FEBS Lett. 2010 May 3; 584(9):1748-59.). In addition, cognitive and cholesterol levels are known to be closely related to schizophrenia (Krakowski M1 and Czobor P, Cholesterol and cognition in schizophrenia: a double-blind study of patients randomized to clozapine, olanzapine and haloperidol. Schizophr Res. 2011 August; 130(1-3):27-33.).

As a specific example, Niemann-pick disease is a rare autosomal recessive hereditary disease in which sphingolipid and cholesterol are accumulated in various organs due to metabolic disorders of sphingolipid to show a variety of clinical symptoms. According to a causal gene and a clinical aspect, the Niemann-pick disease is classified into subtypes of A, B, C, and D. It is known that A and B types are caused by deficiency of sphingomyelinase, and then it is known that C and D types are caused by a transportation disorder of cholesterol. The C type, which shows clinically diverse subacute chronic courses, is known to have a prevalence of about 0.6 to 0.8 person per 100,000 persons according to a report, and a C1 type due to mutation of an NPC1 gene accounts for about 95% of the total. In C type Niemann-pick disease, cholesterol is characteristically accumulated in visceral organs and a nervous system, symptoms are expressed according to the accumulated organs, and the fatality rate is mainly determined by the progression of deposition of a central nervous system. According to recent studies, it was found that sphingosine is a major deposition material for the C type Niemann-pick disease. The C type Niemann-pick disease may show clinically diverse progressions, and its time of onset has been reported variously from newborns to 70s, and a disease period ranges from a few days to 60 years. Hepatosplenomegaly, gait disorders, ocular motility disorders, cognitive disorders, etc. are relatively characteristic, but in the central nervous system, dysmyelination of neurons and degeneration of cerebellar Purkinje cells are caused by selectively invading the cerebellum and the brain stem well to cause related symptoms. It was reported that such a Niemann-pick disease configures the progressive cerebellar ataxia (Timothy J. Maarup et al., Intrathecal 2-Hydroxypropyl-Beta-Cyclodextrin in a Single Patient with Niemann-Pick C1, Mol Genet Metab. 2015 September-October; 116(0): 75-79.).

In the treatment of these neurodegenerative diseases, various candidates have been tested for efficacy as a therapeutic agent in the related art, but limitations have been reported in substantially improving disease symptoms. Therefore, for an effective treatment of degenerative diseases, there is a need for new therapeutic strategies for exhibiting a normalization effect in a practical level.

### DISCLOSURE

### Technical Problem

Therefore, the present inventors had studied a new strategy for a treatment of neurodegenerative diseases, and found that in the case of a combined treatment of stem cells in which VEGF was overexpressed and cyclodextrin in a neurodegenerative disease animal model, a lifespan, a weight, mobility, neurogenic inflammation, neural cell apoptosis, lipid and cholesterol accumulation in various organs including the brain were very effectively improved so that the combined treatment of the two substances had remarkable synergistic effects in a treatment of neurodegenerative diseases, and completed the present invention.

An object of the present invention is to provide a pharmaceutical composition for preventing or treating neurodegenerative diseases comprising, as active ingredients, cyclodextrin or its pharmaceutically acceptable salt; and stem cells in which vascular endothelial growth factor (VEGF) is overexpressed

An object of the present invention is to provide a pharmaceutical composition for preventing or treating neurodegenerative diseases consisting of cyclodextrin or its pharmaceutically acceptable salt; and stem cells in which vascular endothelial growth factor (VEGF) is overexpressed

An object of the present invention is to provide a pharmaceutical composition for preventing or treating neurodegenerative diseases consisting essentially of cyclodextrin or its pharmaceutically acceptable salt; and stem cells in which vascular endothelial growth factor (VEGF) is overexpressed, as active ingredients.

Another object of the present invention is to provide a pharmaceutical complex formulation for preventing or treating neurodegenerative diseases comprising the composition.

Yet another object of the present invention is to provide a use of cyclodextrin or its pharmaceutically acceptable salt; and stem cells in which vascular endothelial growth factor (VEGF) is overexpressed for preparing a pharmaceutical formulation for treating neurodegenerative diseases.

Yet another object of the present invention is to provide a treating method for neurodegenerative diseases comprising administering an effective dose of a composition containing, as active ingredients, cyclodextrin or its pharmaceutically acceptable salt; and stem cells in which vascular endothelial growth factor (VEGF) is overexpressed to a subject requiring the composition.

### Technical Solution

In order to achieve the above objects, the present invention provides a pharmaceutical composition for preventing or treating neurodegenerative diseases containing, as active ingredients, cyclodextrin or its pharmaceutically acceptable salt; and stem cells in which vascular endothelial growth factor (VEGF) is overexpressed

Further, the present invention provides a pharmaceutical composition for preventing or treating neurodegenerative diseases configured by cyclodextrin or its pharmaceutically acceptable salt; and stem cells in which vascular endothelial growth factor (VEGF) is overexpressed.

Further, the present invention provides a pharmaceutical composition for preventing or treating neurodegenerative diseases comprising cyclodextrin or its pharmaceutically acceptable salt; and stem cells in which vascular endothelial growth factor (VEGF) is overexpressed, which are essentially configured as active ingredients.

In order to achieve another object of the present invention, the present invention provides a pharmaceutical complex formulation for preventing or treating neurodegenerative diseases comprising the composition.

In order to achieve another object of the present invention, the present invention provides a use of cyclodextrin or its pharmaceutically acceptable salt; and stem cells in which vascular endothelial growth factor (VEGF) is overexpressed for preparing a pharmaceutical formulation for treating neurodegenerative diseases.

In order to achieve another object of the present invention, the present invention provides a treating method for neurodegenerative diseases comprising administering an effective dose of a composition containing, as active ingredients, cyclodextrin or its pharmaceutically acceptable salt; and stem cells in which vascular endothelial growth factor (VEGF) is overexpressed to a subject requiring the composition.

Hereinafter, the present invention will be described in detail.

The present inventors found that in the case of a combined treatment of cyclodextrin and stem cells in which VEGF is overexpressed, effects of a lifespan increase, mobility improvement, inhibition of neurogenic inflammation, inhibition of neural cell apoptosis and inhibition of lipid accumulation in organs including the brain in a neurodegenerative disease model were remarkably excellent as compared to an effect of cyclodextrin or VEGF alone. These synergistic effects by the combined treatment of cyclodextrin and stem cells in which VEGF is overexpressed are first published in the present invention.

Accordingly, the present invention provides a pharmaceutical composition for preventing or treating neurodegenerative diseases containing cyclodextrin or its pharmaceutically acceptable salt; and stem cells in which VEGF is overexpressed, as active ingredients.

Further, the present invention provides a pharmaceutical composition for preventing or treating neurodegenerative diseases consisting of cyclodextrin or its pharmaceutically acceptable salt; and stem cells in which VEGF is overexpressed.

Further, the present invention provides a pharmaceutical composition for preventing or treating neurodegenerative diseases, comprising cyclodextrin or its pharmaceutically acceptable salt; and stem cells in which VEGF is overexpressed, which are essentially constituted as active ingredients.

In the present invention, the ‘cyclodextrin (abbreviated as CD)’ refers to an oligosaccharide in which glucose molecules form a ring shape by a α-1,4 glycosidic bond. In the present invention, the cyclodextrin means including at least one selected from the group consisting of α-cyclodextrin, β-cyclodextrin, and γ-cyclodextrin, and in the present invention, means including all derivatives (particularly, a sulfobutylether group or a hydroxypropyl substituent) of the cyclodextrin. For example, so long as cyclodextrins are known in the art, a type of derivative is not particularly limited, but preferably, types which have been used for neurodegenerative diseases in the related art include 2-hydroxypropyl-α-cyclodextrin, sulfobutylether-α-cyclodextrin, 2-hydroxypropyl-β-cyclodextrin, sulfobutylether-β-cyclodextrin, methyl-β-cyclodextrin, 2-hydroxypropyl-γ-cyclodextrin, sulfobutylether-γ-cyclodextrin, etc. More preferably, the cyclodextrin of the present invention may be hydroxypropylated cyclodextrin, and is not specifically limited thereto, but includes 2-hydroxypropyl-α-cyclodextrin, 2-hydroxypropyl-β-cyclodextrin, or 2-hydroxypropyl-γ-cyclodextrin. Most specifically, the cyclodextrin of the present invention may be 2-hydroxypropyl-β-cyclodextrin.

The cyclodextrin of the present invention may be used itself or in a form of a pharmaceutically acceptable salt. In the present invention, the term ‘pharmaceutically acceptable’ refers to non-toxicity that does not inhibit a pharmacological action of the active ingredient, is physiologically acceptable, and does not normally cause an allergic reaction such as gastrointestinal disorders and dizziness or a similar reaction thereto when administered to humans. The salt is not limited thereto, but may be an acid addition salt formed by pharmaceutically acceptable free acid. The free acid may use organic acid and inorganic acid. The organic acid is not limited thereto, but includes citric acid, acetic acid, lactic acid, tartaric acid, maleic acid, fumaric acid, formic acid, propionic acid, oxalic acid, trifluoroacetic acid, benzoic acid, gluconic acid, metasulfonic acid, glycolic acid, succinic acid, 4-toluenesulfonic acid, glutamic acid, and aspartic acid. Further, the inorganic acid is not limited, but includes hydrochloric acid, bromic acid, sulfuric acid, and phosphoric acid.

The cyclodextrin or its pharmaceutically acceptable salt of the composition according to the present invention may use salts which are isolated from the nature, prepared by a chemical synthetic method known in the art, or commercially sold.

In the present invention, containing, as an active ingredient, the stem cells in which the VEGF is overexpressed means containing all of stem cell cultures containing the stem cells or concentrates of the cultures as an active ingredient.

The ‘vascular endothelial growth factor (VEGF)’ refers to glycoprotein of 34-42 kDa as a growth factor which selectively acts on vascular endothelial cells. The VEGF of the present invention may be used by those skilled in the art to appropriately select a specific sequence depending on a biological subject to be applied so long as it is VEGF known in the art and is not limited thereto, but includes all of VEGFA, VEGFB, VEGFC, VEGFD, VEGFE or VEGFF and includes all of full-length (VEGF-total) proteins thereof and VEGF-121, VEGF-165, VEGF-189 or VEGF-206 as a splicing variant form. Although not limited thereto, but as the human VEGF protein sequence, NCBI (Genebank) Reference Sequence: NP_001020537.2, NP_003367.4 NP_001020538.2, NP_001020539.2, NP_001020540.2, NP_001020541.2, NP_001028928.1 or NP_001165093.1 is known in the art, and in the present invention, these full-length sequences, or active fragments thereof (i.e., splice variants) may be used without limitations. In the present invention, it may be preferred to use VEGFA as human (Homo sapiens) VEGF, and its full-length sequence or active fragments (i.e., splice variants) may be used without limitations.

In the present invention, the VEGF includes its functional equivalent. The functional equivalent has sequence homology with the sequence of at least 70% or more, preferably 80% or more, more preferably 90% or more, and much more preferably 95% or more as a result of addition, substitution or deletion of amino acids to the aforementioned known VEGF amino acid sequences, and refers to a protein having substantially the same activity as the aforementioned known VEGF.

The term ‘stem cells’ used in the present specification are undifferentiated cells with the ability to differentiate into various tissues, which may be classified into totipotent stem cells, pluripotent stem cells, and multipotent stem cells.

In the present invention, according to an origin or a type thereof, the stem cells may be adult stem cells, embryonic stem cells, mesenchymal stem cells, tumor stem cells or induced pluripotent stem cells. In addition, the adult stem cells may be neural stem cells or neural progenitor cells.

The neural stem cell (NSC) is a cell which can be self-renewal and has differentiation potency into nervous system cells, and the NSC is a cell which may be differentiated into a neuron, an astrocyte, and an oligodendrocyte.

In addition, the term ‘mesenchymal stem cell (MSC)’ as used herein is a multipotent stem cell which has the ability to differentiate into ectoderm cells, such as various mesodermal cells including bone, cartilage, fat, and muscle cells or ectoderm cells such as neurons. The mesenchymal stem cells may be derived from one selected from the group consisting of umbilical cord, umbilical cord blood, bone marrow, fat, muscle, nerve, skin, amniotic membrane, chorion, decidual membrane, and placenta, but is not limited thereto. In addition, the mesenchymal stem cells may be derived from humans, fetus, or mammals other than humans. Mammals other than humans may be more preferably dogs, cats, monkeys, cow, sheep, pig, horse, rat, mouse or guinea pig, and the origin is not limited.

The stem cells may be isolated and obtained from animals (particularly, mammals). Since a specific marker is known for each stem cell, those skilled in the art may selectively isolate and obtain only stem cells by using the specific marker as a marker. For example, NCAM, Nestin, Tuj 1, and Sox2 are known as neural stem cell markers in the art, those skilled in the art may selectively isolate and obtain only stem cells by using the neural stem cell markers as a marker.

The stem cells in which VEGF is overexpressed of the present invention may be transformed by a recombinant vector including a polynucleotide (e.g., GenBank ID: NM_001025366.2, NM_003376.5, NM_001025367.2, NM_001025368). 0.2, NM_001025369.2, NM_001025370.2, NM_001033756.2, or NM_001171622.1) encoding the VEGF. Since the recombinant vector should be able to overexpress VEGF encoding nucleic acids in stem cells, the recombinant vector is preferably a recombinant expression vector form. The recombinant expression vector may be prepared by operably linking a VEGF encoding nucleic acid and a regulatory sequence (e.g., a promoter, a secretion sequence, an enhancer, an upstream activating sequences, a transcription termination factor, etc.) capable of exhibiting functions in stem cells (particularly, neural cells) of a target organism (e.g., mammalian animal) to a commercially available basic vector (i.e., a backbone vector). The ‘operably linked’ means linked by a method of enabling the expression of the nucleic acid when an appropriate nucleic acid molecule is linked to an expression regulatory sequence. The recombinant expression vector may include a selection marker, and may be used by appropriately selecting a method known in the art. For example, as the selection marker, antibiotic resistance genes such as a kanamycin resistance gene and a neomycin resistance gene, and fluorescent proteins such as a green fluorescent protein and a red fluorescent protein are included, but it is not limited thereto.

The transformation may proceed according to known methods, and includes calcium phosphate transfection, electrophoresis, transduction, (DEAE-dextran mediated transfection, microinjection, cationic lipid-transfection, ballistic introduction, and the like, but is not limited thereto.

In one embodiment of the present invention, as VEGF-overexpressed stem cells for administration to a neurodegenerative disease mouse model, neuronal stem cells have been isolated and obtained from VEGFtg mice overexpressing brain cell-specific VEGF. The VEGFtg mice are mice transformed so that VEGF is overexpressed specifically only to neural (stem) cells using a recombinant vector (plasmid) including a neuron-specific enolase (NSE) promoter and a VEGF encoding nucleic acid, and the transformation method may refer to the following documents: Yaoming Wang et al., VEGF overexpression induces post-ischaemic neuroprotection, but facilitates haemodynamic steal phenomena, Brain (2005), 128, 52-63. Specifically, the present inventors used the VEGFtg mice used in the Yaoming Wang et al., (2005) document in Examples, and the mice express human VEGFA165 specifically to neural (stem) cells. The human VEGFA165 polypeptide, for example, is known in the art as having an amino acid sequence such as NCBI Reference Sequence: NP_001165097.1, but is not limited thereto. In the present invention, a functional equivalent thereof may be used without limitation. The VEGFA165 polypeptide may be encoded by a polynucleotide such as NM_001171626.1, but is not limited thereto.

The stem cells in which the VEGF is overexpressed may be administered simultaneously, separately, or sequentially with cyclodextrin or its pharmaceutically acceptable salt.

The cyclodextrin (or its pharmaceutically acceptable salt) and the stem cells in which the VEGF is overexpressed, which are the active ingredients of the present invention, may be included together in a pharmaceutical formulation and administered simultaneously through the same administration site, or provided as a separate formulation to be administered simultaneously or sequentially through different administration sites.

Specifically, ‘simultaneous administration’ means administration of the two active ingredients together through the same route of administration, or administration of the two active ingredients through the same or different routes of administration, respectively, at substantially the same time (e.g., at an interval of administration of 15 minutes or less). The separate administration means administration of the two active ingredients through the same or different routes of administration at a regular time interval (for example, every three days). The sequential means administration of the two active ingredients through the same or different routes of administration with a certain order rule according to a disease condition of the patient.

The route of administration may be oral or parenteral administration. The parenteral administration method is not limited thereto, but may be intravenous, intramuscular, intraarterial, intramedullary, intrathecal, intracardiac, transdermal, subcutaneous, intraneural, intraventricular (subventricular zone), intracerebrovascular, intraperitoneal, intranasal, intestinal canal, topical, sublingual, or intrarectal administration.

Preferably, the route of administration of cyclodextrin in the pharmaceutical composition of the present invention may be subcutaneous, intravenous, arterial or intraventricular (subventricular zone) injection, and most preferably, the stem cells in which the VEGF is overexpressed in the pharmaceutical composition of the present invention may be injected into a subventricular zone (SVZ). The present inventors have proved that a medicinal effect by improvement of an SVZ environment could not be achieved by introducing VEGF alone into SVZ or by introducing normal neural stem cells (non-genetically modified wild-type individual-derived neural stem cells) into SVZ, but the effect was exhibited only when a form of neural stem cells in which the VEGF was overexpressed was administered and filed the fact (Application No. 10-2017-0015676).

The pharmaceutical composition according to the present invention may include only a pharmaceutically effective dose of cyclodextrin (or its pharmaceutically acceptable salt) and stem cells in which VEGF is overexpressed, or may additionally include a pharmaceutically acceptable carrier. The ‘pharmaceutically effective dose’ refers to a dose that shows a higher response than a negative control group, preferably, means a sufficient dose which exhibits effects of a lifespan increase, mobility improvement, inhibition of neurogenic inflammation, inhibition of neural cell apoptosis and inhibition of lipid accumulation in organs including the brain by administering a combination of the two active ingredients in the treatment or prevention of neurodegenerative diseases.

Specifically, a pharmaceutically effective dose of the cyclodextrin or its pharmaceutically acceptable salt included as an active ingredient in the pharmaceutical composition of the present invention is a dose which is administered with a daily dose of 50 mg/day/kg of body weight to 4000 mg/day/kg of body weight. More specifically, the pharmaceutically effective dose may be a dose administered with 100 mg/day/kg of body weight to 4000 mg/day/kg of body weight.

In addition, the pharmaceutically effective dose of the stem cells in which the VEGF is overexpressed included as an active ingredient in the pharmaceutical composition of the present invention is characterized as a dose administered with a daily dose of 1×10⁵ cells/day to 1×10⁶ cells/day. More specifically, the pharmaceutically effective dose may be a dose administered with 5×10⁵ cells/day to 1×10⁶ cells/day.

However, the pharmaceutically effective dose may be properly changed according to a disease and its severity, an age, a weight, a health condition, and a gender of a patient, an administration route, a treatment period, and the like.

In the present invention, the ‘neurodegenerative disease’ means a disease caused by the death or dysfunction of neural cells constituting the central nervous system, and the neurodegenerative diseases have common characteristics such as death of neural cells such as brain cells, brain capacity reduction, and neurogenic inflammation, and particularly, are known to be closely related to changes in cholesterol and lipid metabolism. Therefore, so long as the diseases are known in the art as neurodegenerative diseases, specific types thereof are not limited in the present invention, and a type known to be very closely related to changes in cholesterol and lipid metabolism may be more preferable. For example, the neurodegenerative diseases may be at least one selected from the group consisting of Neiman-pick disease, Alzheimer's disease, Parkinson's disease, Huntington's disease, Lou Gehrig's disease, schizophrenia, Gaucher disease, Fabry disease, Tay-Sachs disease, Sandhoff disease and cerebellar ataxia.

Most preferably, the neurodegenerative disease may be Neiman-pick disease or and cerebellar ataxia. In the present invention, the Neiman-pick disease is a disease in which lipids are accumulated in reticuloendothelial cells, which corresponds to a genetic disease. A type of the Neiman-pick disease of the present invention is not limited, and for example, the Neiman-pick disease may be A-type, B-type, C-type, D-type, E-type or F-type Neiman-pick disease. Particularly, the Neiman-pick disease of the present invention may be C-type Neiman-pick disease. The C-type Neiman-pick disease is a genetic disease that causes various neurological disorders, such as memory and intelligence disorders due to the accumulation of sphingolipid and cholesterol in cells due to metabolic disorders of lipids, which are a major organic substance that constitutes a living body, together with proteins and sugars.

In the present invention, the cerebellar ataxia refers to a neurological disorder with a symptom in which movement is poor and coordination between the movements is not made due to dysfunction of the cerebellum and includes all cerebellar ataxias caused by various medical and neurological diseases or genetic predispositions.

According to an embodiment of the present invention, the combined administration of the neural stem cells in which the VEGF is overexpressed and the cyclodextrin exhibits remarkably synergistic effects to improve a lifespan, a weight, mobility, neurogenic inflammation, neural cell apoptosis, and lipid and cholesterol accumulation in various organs including the brain of mice in a neurodegenerative disease mouse model. In addition, although the neural stem cells in which the VEGF is overexpressed are administered to the subventricular zone and the cyclodextrin is injected subcutaneously, the combined administration causes results that damage to neural cells is prevented and the inflammatory response is alleviated in the cerebellum. As described above, it has been confirmed that the combined administration is very excellent in effects of preventing or treating Neiman-pick disease, Alzheimer's disease, Parkinson's disease, Huntington's disease, Lou Gehrig's disease, Schizophrenia, Gaucher disease, Fabry disease, Tay-Sachs disease, Sandhof's disease, and cerebellar ataxia, which are known to be closely related to changes in cholesterol and lipid metabolism in the related art.

In the present invention, the ‘treatment’ comprehensively refers to improvement of symptoms of neurodegenerative diseases or diseases related to neurodegenerative diseases, which may include treatment (becoming substantially the same condition as a normal subject) or substantial prevention (inhibiting or delaying the onset of diseases) for these diseases, or alleviating conditions thereof (symptoms are improved or beneficially changed), and include alleviating, treating or preventing a symptom or most of symptoms derived from neurodegenerative diseases or diseases related to neurodegenerative diseases, but is not limited thereto.

The pharmaceutical composition of the present invention may be variously formulated according to a route of administration by a method known in the art, together with a pharmaceutically acceptable carrier to exhibit the synergistic effect by using a combination of the cyclodextrin (or its pharmaceutically acceptable salt) and the stem cells in which the VEGF is overexpressed. The ‘pharmaceutically acceptable’ generally means a non-toxic composition which is physiologically acceptable, but does not inhibit actions of the active ingredients when administered to the human, and does not cause an allergic reaction such as gastroenteric trouble and dizziness or a similar reaction thereto. The carrier includes all kinds of solvents, dispersion media, oil-in-water or water-in-oil emulsions, aqueous compositions, liposomes, microbeads and microsomes. The pharmaceutically acceptable carrier to be contained in the pharmaceutical composition is generally used in preparation and includes lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil, but is not limited thereto. Other pharmaceutically acceptable carriers may refer to carriers disclosed in the following document (Remington's Pharmaceutical Sciences, 19th ed., Mack Publishing Company, Easton, Pa., 1995).

The pharmaceutical composition may further include a lubricant, a wetting agent, a sweetening agent, a flavoring agent, an emulsifying agent, a suspending agent, a preservative, and the like in addition to the above ingredients. Specifically, in oral administration, a binder, a lubricant, a disintegrant, an excipient, a solubilizer, a dispersant, a stabilizer, a suspending agent, a pigment, and a perfume may be used. In the case of injections, a buffering agent, a preservative, a painless agent, a solubilizer, an isotonic agent, and a stabilizer may be mixed and used. In the case of topical administration, a base, an excipient, a lubricant, and a preservative may be used.

In addition, the composition of the present invention may be used in the form of a general pharmaceutical formulation. Parenteral formulations may be prepared in forms of sterile aqueous solutions, non-aqueous solvents, suspensions, emulsions or lyophilized preparations, injections, transdermal injections, nasal inhalations, and the like. In oral administration, the composition may be prepared in a form of tablets, troches, capsules, elixirs, suspensions, syrups, or wafers. Injections may be prepared in unit dosage ampoules or in multiple dosage forms. The injections must be sterile and protected from contamination of microorganisms such as bacteria and fungi. Examples of suitable carriers for injections may include, but are not limited to, solvents or dispersion media including water, ethanol, polyols (e.g., glycerol, propylene glycol, and liquid polyethylene glycols), mixtures thereof and/or vegetable oils. More preferably, as suitable carriers, a Hanks' solution, a Ringer's solution, a phosphate buffered saline (PBS) containing triethanol amine or sterile water for injection, and an isotonic solution such as 10% ethanol, 40% propylene glycol and 5% dextrose may be used. In order to protect the injection from microbial contamination, various antibacterial and antifungal agents such as paraben, chlorobutanol, phenol, sorbic acid, thimerosal, and the like may be further included. In addition, the injection may further include an isotonic agent, such as sugar or sodium chloride, in most cases.

In addition, the pharmaceutical composition of the present invention may be administered by any device capable of transferring an active ingredient to a target cell. Preferred methods and formulations of administration are intravenous injections, subcutaneous injections, intradermal injections, intramuscular injections, or drop injections. The injections may be prepared by using aqueous solvents such as a PBS or a ringer solution, and non-aqueous solvents such as vegetable oils, higher fatty acid esters (e.g., ethyl oleate), and alcohols (e.g., ethanol, benzyl alcohol, propylene glycol, or glycerin) and may include a pharmaceutical carrier such as a stabilizer (e.g., ascorbic acid, sodium hydrogen sulfite, sodium pyrosulfite, BHA, tocopherol, EDTA, etc.), an emulsifier, a buffer for pH control, and a preservative to prevent microbial growth (e.g., phenyl mercury nitrate, thimerosal, benzalkonium chloride, phenol, cresol, benzyl alcohol, etc.) for the prevention of degeneration. A method for treating or preventing neurodegenerative diseases using the composition of the present invention includes administering an effective dose (pharmaceutically effective dose) of the therapeutic composition of the present invention to a subject in need thereof. The pharmaceutically effective dose may be easily determined by those skilled in the art according to factors well-known in the medical field, such as a type of disease, age, a weight, health, and gender of a patient, sensitivity to a drug of a patient, a route of administration, a method of administration, a frequency of administration, a duration of treatment, and drugs to be combined or used simultaneously.

Further, the pharmaceutical composition of the present invention may be formulated by using a method known in the art so as to provide rapid, sustained, or delayed release of the active ingredient after being administrated to a mammal.

Further, the present invention also provides a pharmaceutical complex formulation for preventing or treating neurodegenerative diseases including the pharmaceutical composition.

The pharmaceutical complex formulation of the present invention may be formulated such that the cyclodextrin and the stem cells in which the VEGF is overexpressed as constitute elements are simultaneously included in one formulation, depending on a method of administration and a route of administration, and each constitute element may be individually formulated and included in one package, depending on a dosage unit, such as daily or one time. The formulations individually formulated by the cyclodextrins and the stem cells in which the VEGF is overexpressed may be the same or not. A specific formulation method of the pharmaceutical complex formulation of the present invention and a pharmaceutically acceptable carrier that may be included in the formulation are the same as those described in the pharmaceutical composition and may refer to the following document (Remington's Pharmaceutical Sciences, 19th ed., Mack Publishing Company, Easton, Pa., 1995). Preferably, the formulation of the present invention may be an injection.

The cyclodextrin (or its pharmaceutically acceptable salt) and the stem cells in which the VEGF is overexpressed, which are constituent elements of the pharmaceutical complex formulation according to the present invention, may be administered simultaneously or separately or in a predetermined order (sequentially). The ‘simultaneous administration’ means administration of the two active ingredients together through the same route of administration, or administration of the two active ingredients through the same or different routes of administration, respectively, at substantially the same time (e.g., at an interval of administration of 15 minutes or less). The separate administration means administration of the two active ingredients through the same or different routes of administration at a regular time interval (for example, every three days). The sequential administration means administration of the two active ingredients through the same or different routes of administration with a certain order rule according to a disease condition of the patient. The complex formulation may be formulated to include fully a daily dose in one dose, but may be formulated to be divided and administered into two, three, four, etc. per day.

A preferable dose of the pharmaceutical complex formulation of the present invention may be properly changed according to various factors such as a disease and its severity, an age, a weight, a health condition, and a gender of a patient, a route of administration, a treatment period, and the like. Since the bioavailability of the pharmaceutically active ingredient has an individual difference, it may be preferred to confirm the blood concentration of each drug by an assay based on a monoclonal antibody known in the art at the beginning of the administration of the pharmaceutical formulation of the present invention.

The present invention provides a use of cyclodextrin or its pharmaceutically acceptable salt; and stem cells in which VEGF is overexpressed for preparing a pharmaceutical formulation for treating neurodegenerative diseases.

The present invention provides a treating method for neurodegenerative diseases including administering an effective dose of a composition containing, as active ingredients, cyclodextrin or its pharmaceutically acceptable salt; and stem cells in which VEGF is overexpressed to a subject requiring the composition.

The term ‘effective dose’ of the present invention means an amount which exhibits effects of improving, treating, preventing, detecting, diagnosing of neurodegenerative diseases, or inhibiting or alleviating neurodegenerative diseases when administered to the subject. The ‘subject’ may be animals, preferably, mammals, particularly animals including humans and may also be cells, tissues, and organs derived from animals. The subject may be a patient requiring the effects.

The term ‘treatment’ of the present invention comprehensively refers to improving neurodegenerative diseases or symptoms of neurodegenerative diseases, and may include treating or substantially preventing these diseases, or improving the conditions thereof and includes alleviating, treating or preventing a symptom or most of symptoms derived from neurodegenerative diseases, but is not limited thereto.

The term ‘comprising’ of the present invention is used in the same manner as ‘containing’ or ‘characterizing’, and does not exclude additional ingredients or steps of the method which are not mentioned in the composition or the method. The term ‘consisting of’ means excluding additional elements, steps or ingredients, etc., unless otherwise noted. The term ‘essentially consisting of’ means including ingredients or steps that do not substantially affect basic properties thereof in addition to the described ingredients or steps within the scope of the composition or the method.

### Advantageous Effects

A combined treatment of cyclodextrin and stem cells in which VEGF is overexpressed has remarkable synergistic effects, with respect to therapeutic efficacy on the diseases, such as a lifespan increase, mobility improvement, inhibition of neurogenic inflammation, inhibition of neural cell apoptosis and inhibition of lipid accumulation in organs including the brain, in a neurodegenerative disease model.

### DESCRIPTION OF DRAWINGS

FIG. 1 illustrates an administration schedule and an administration method of each test substance used in the present test to confirm an increase in brain-specific VEGF (particularly, injection of VEGF-overexpressed stem cells of the present invention and effects of combined administration of cyclodextrin. NP-C mice were administered with cyclodextrin (4000 mg/kg, once a week, subcutaneous injection) alone or administered with a combination of VEGF-overexpressed neural stem cells (VEGFtg NSC, 10⁶ cells/3 ul, twice a week, intraventricular injection) and cyclodextrin and brain cell-specific VEGF-overexpressed NP-C mice were administered with cyclodextrin (4000 mg/kg, once a week, subcutaneous injection).

FIG. 2 illustrates results of confirming whether a survival rate is increased for each age after administration of cyclodextrin alone or combined administration in ventricles of VEGF-overexpressed neural stem cells to NP-C mice, and after administration of cyclodextrin to VEGFNP-C mice (n=8 to 10 per group). *p<0.05, **p<0.01 data were illustrated as mean±s.e.m.

FIG. 3 illustrates results of confirming whether a weight is changed for each age after administration of cyclodextrin alone or combined administration in ventricles of VEGF-overexpressed neural stem cells to NP-C mice, and after administration of cyclodextrin to VEGFNP-C mice (n=8 to 10 per group). *p<0.05, **p<0.01 data were illustrated as mean±s.e.m.

FIG. 4 illustrates results of confirming whether mobility is improved for each age through a Rota-rod test after administration (subcutaneous injection) of cyclodextrin alone or combined administration (intraventricular injection) of VEGF-overexpressed neural stem cells to NP-C mice, and after administration (subcutaneous injection) of cyclodextrin to VEGFNP-C mice (n=8 to 10 per group). *p<0.05 data were illustrated as mean±s.e.m.

FIG. 5 illustrates results of confirming whether mobility is improved for each age through a Beam test using a bar of a width of 12 mm (FIG. 5A) or a bar of a width of 6 mm (FIG. 5B) after administration (subcutaneous injection) of cyclodextrin alone or combined administration (intraventricular injection) of VEGF-overexpressed neural stem cells to NP-C mice, and after administration (subcutaneous injection) of cyclodextrin to VEGFNP-C mice (n=8 to 10 per group). *p<0.05 data were illustrated as mean±s.e.m.

FIGS. 6A and 6B illustrate microscopic images (FIG. 6A) and quantitative results (FIG. 6B), as results of confirming an inflammatory response through GFAP (astroctyte) staining by extracting the cortex of mice when the mice are 10-week-old after administration (subcutaneous injection) of cyclodextrin alone or combined administration (intraventricular injection) of VEGF-overexpressed neural stem cells to NP-C mice, and after administration (subcutaneous injection) of cyclodextrin to VEGFNP-C mice (n=3 per group). *p<0.05, **p<0.01 data were illustrated as mean±s.e.m.

FIGS. 7A and 7B illustrate microscopic images (FIG. 7A) and quantitative results (FIG. 7B), as results of confirming an inflammatory response through GFAP (astroctyte) staining by extracting the cerebellum of mice when the mice are 10-week-old after administration (subcutaneous injection) of cyclodextrin alone or combined administration (intraventricular injection) of VEGF-overexpressed neural stem cells to NP-C mice, and after administration (subcutaneous injection) of cyclodextrin to VEGFNP-C mice (n=3 per group). *p<0.05, **p<0.01 data were illustrated as mean±s.e.m.

FIGS. 8A and 8B illustrate microscopic images (FIG. 8A) and quantitative results (FIG. 8B), as results of confirming a degree of reduction of neural cells (particularly, Purkinje cells) existing in a Purkinje cell layer through Calbindin staining by extracting the cerebellum of mice when the mice are 10-week-old after administration (subcutaneous injection) of cyclodextrin alone or combined administration (intraventricular injection) of VEGF-overexpressed neural stem cells to NP-C mice, and after administration (subcutaneous injection) of cyclodextrin to VEGFNP-C mice (n=3 per group, molecular layer (ML), Purkinje cell layer (PCL), granular cell layer (GCL)). *p<0.05, **p<0.01 data were illustrated as mean±s.e.m.

FIGS. 9A and 9B illustrate results of confirming lipid accumulation degrees of Sphingosine (FIG. 9A) and Sphingomyelin (FIG. 9B) by extracting the cortex, the cerebellum, the liver, the lung, the kidney, and the spleen of mice when the mice are 10-week-old after administration (subcutaneous injection) of cyclodextrin alone or combined administration (intraventricular injection) of VEGF-overexpressed neural stem cells to NP-C mice, and after administration (subcutaneous injection) of cyclodextrin to VEGFNP-C mice (n=3 per group). *p<0.05, **p<0.01, ***p<0.001 data were illustrated as mean±s.e.m.

FIGS. 10A and 10B illustrate diagrams of confirming whether to accumulate cholesterol by Amplex red assay (Unesterified cholesterol, FIG. 10A) and quantification after Filipin staining (FIG. 10B) by extracting the cortex, the cerebellum, the liver, the lung, the kidney, and the spleen of mice when the mice are 10-week-old after administration (subcutaneous injection) of cyclodextrin alone or combined administration (intraventricular injection) of VEGF-overexpressed neural stem cells to NP-C mice, and after administration (subcutaneous injection) of cyclodextrin to VEGFNP-C mice (n=3 per group). *p<0.05, **p<0.01, ***p<0.001 data were illustrated as mean±s.e.m.

### MODES OF THE INVENTION

Hereinafter, the present invention will be described in detail.

However, the following Examples are just illustrative of the present invention, and the contents of the present invention are not limited to the following Examples.

Test Materials and Test Methods

1. Preparation of Mice

In Balb/C (Orient, wild type), NPC mutant mice in which an NPC1 gene is deleted (RIKEN, received from Japan, NP-C mice; a weight was smaller than that of normal mice with the same week old, a serious mobility loss was shown from 4 to 6 week old to show tremors and seizures of the limbs, and a lifespan was approximately 9 to 10 weeks), and brain cell-specific VEGF-overexpressed NP-C mice (VEGFNP-C mice; produced through mating of VEGFtg mice overexpressing VEGF in a brain cell-specific promoter (received from University of Heidelberg, Germany, Yaoming Wang et al., (2005)) and NP-C mice, a lifespan was approximately 9 to 10 weeks like the NP-C mice, and brain inflammation and lipid accumulation were alleviated as compared to the NP-C mice), systems thereof were kept by genotyping through PCR. The mice were disposed in a test group by using a block randomization method. In order to eliminate prejudice, data collection and data analysis were never involved. All mice tests were approved by Kyungpook National University Institutional Animal Care and Use Committee (IACUC).

2. Culture of Neurosphere and Culture of VEGF-Overexpressed Neural Stem Cells

In order to obtain neural stem cells overexpressing VEGF, the subventricular zones of VEGFtg mice were extracted from the mouse brain. Each tissue was extracted from an ice-cold Hibernate A/B27/Glutamax medium (HABG) (Invitrogen) and then immersed in a papain (Worthington) solution and reacted for 30 minutes at 37° C. to decompose the tissue. Subsequently, the decomposed tissue was centrifuged using an Optiprep (Sigma) density gradient solution, and a layer containing neural stem cells was separated, and then cultured for 1 week in a Neurobasal A (Invitrogen)/B27 medium containing glutamax (0.5 mM), gentamycin (10 ug/ml, Invitrogen), mouse fibroblast growth factor 2 (mFGF2, 5 ng/ml, Invitrogen), and mouse platelet-derived growth factor-bb (mPDGFbb, 5 ng/ml, Invitrogen). The cultured neural stem cells were grown into spherical neurosphere, and after 1 week, each cell was separated with a Triple select (Gibco) solution and then and injected into single cells. In order to confirm a successful yield of neural stem cells overexpressing VEGF, the expression thereof was confirmed by staining using a neural stem cell marker, Nestin antibody (milipore, MAB353).

3. Injection of Cyclodextrin and VEGF-Overexpressed Neural Stem Cells

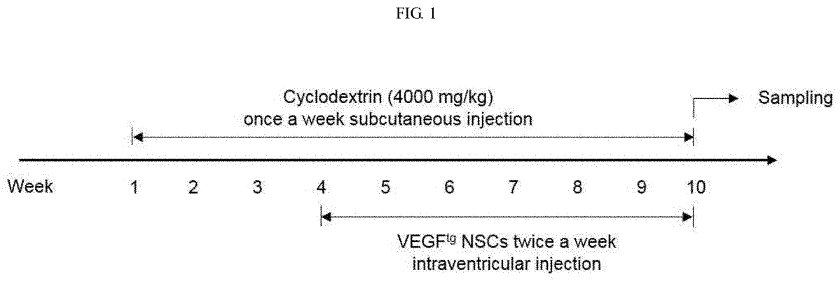

Administration of cyclodextrin (Sigma, H107) and VEGF-overexpressed neural stem cells was performed on a schedule as illustrated in FIG. 1. Specifically, NP-C mice or VEGFNP-C mice were subcutaneously injected with cyclodextrin by 4000 mg/kg from 1 week old. When the NP-C mice injected with the cyclodextrin were 4-week-old, a cannula was mounted in a subventricular zone (SVZ) for combined injection into the SVZ of the VEGF-overexpressed neural stem cells, and the VEGF-overexpressed neural stem cells (to 1×10⁶ cell/3 ul) were injected into the SVZ twice a week. A cell injection rate was implanted at a flow rate of 0.3 μl/min.

4. Immunofluorescent Staining

The brain tissue (cortex or cerebellum) of a mouse was cut to a thickness of 50 μm using vibratome, and then cultured with anti-GFAP (rabbit, 1:500, DAKO) and anti-Calbindin (rabbit, 1:100, milipore). In addition, filipin staining (Polysciences, Inc.) was performed on tissues of each organ from the mouse to confirm the accumulation degree of cholesterol. Cortex, cerebellum, liver, lung, kidney, and spleen sites were analyzed using a laser scanning confocal microscope or an Olympus BX51 microscope equipped with Fluoview SV1000 imaging software (Olympus FV1000, Japan). A percentage of an area of a stained area to an area of total tissues was quantified using Metamorph software (Molecular Devices).

5. Measurement of Lipid

The cortex, the cerebellum, and organs (liver, lung, kidney, spleen, etc.) of each mouse were extracted, added with a homogeneous buffer solution containing 50 mM HEPES (Invitrogen), a 150 mM sodium chloride solution (NaCl) (Sigma), 0.2% Igepal CA-630 (Sigma), and a protease inhibitor (Milipore), homogenized using a homogenizer, and then centrifuged at 13,000 rpm for 10 minutes. After 10 minutes, additional centrifugation was performed at 13,000 rpm for 30 minutes. After 30 minutes, a supernatant was taken, added with DCM (dichloromethane, Sigma), DCM:M (dichloromethane:methanol=1:3, Sigma), and 10% sodium hypochlorite (NaHCl) in sequence, and centrifuged at 13,000 rpm for 1 minute, and then only an organic soluble layer was separated. The separated sample was dried using a rotary vacuum evaporator. A dry lipid extract obtained above was resuspended in 0.2% Igepal CA-630 (Sigma), and then the concentration of each lipid was measured using a UPLC system.

6. Measurement of Cholesterol

The cortex, the cerebellum, and organs (liver, lung, kidney, spleen, etc.) of each mouse were extracted, added with a homogeneous buffer solution containing a 50 mM phosphate buffer, a 500 mM sodium chloride solution (NaCl), 25 mM cholic acid, and 0.5% Triron X-100, homogenized using a homogenizer, and then centrifuged at 13,000 rpm for 10 minutes. After 10 minutes, only an organic soluble layer was separated and a cholesterol level was measured using an Amplex Red Cholesterol Assay Kit (Molecular Probes).

7. Sensory Ability Test

In order to confirm the mobility of a mouse in each test group, Rota-rod and Beam tests were performed. In the Rota-rod test (Ugo Basile, Comerio, VA, Italy), a Rota-rod movement was performed three times or more at a rotation rate of 4 rpm using a machine equipped with a rod of a diameter of 3 cm which was appropriately processed for providing a grip, and then an endurance time of a test animal was measured in units of second and an average value thereof was recorded. Each Rota-rod movement test did not exceed 5 minutes per one time. In the Beam test, a mouse was placed on a starting point of a rod of a width of 6 mm or 12 mm and then a time taken to move to an end point was measured.

8. Statistical Analysis

Comparison with each group was performed using a Student's t-test. In the case of comparing two or more groups with another group, a one way ANOVA and a Tukey's HSD test were performed. Comparison of overall survival was performed using a Log-rank test. All statistical analyses were performed using SPSS statistical software. p<0.05, p<0.01, and p<0.001 were considered to be significant

### Example 1: Confirmation of Survival Rate and Change in Weight of NP-C Mice by Brain-Specific VEGF Increase and Administration of Cyclodextrin

1-week-old NP-C mice were administered with cyclodextrin (4000 mg/kg, once a week, subcutaneous injection) alone or a combination of VEGF-overexpressed neural stem cells (10⁶ cell/3 ul, twice a week, intraventricular injection) and cyclodextrin. Brain cell-specific VEGF-overexpressed NP-C mice (VEGFNP-C) were administered with cyclodextrin (4000 mg/kg, once a week, subcutaneous injection) from 1 week old (FIG. 1).

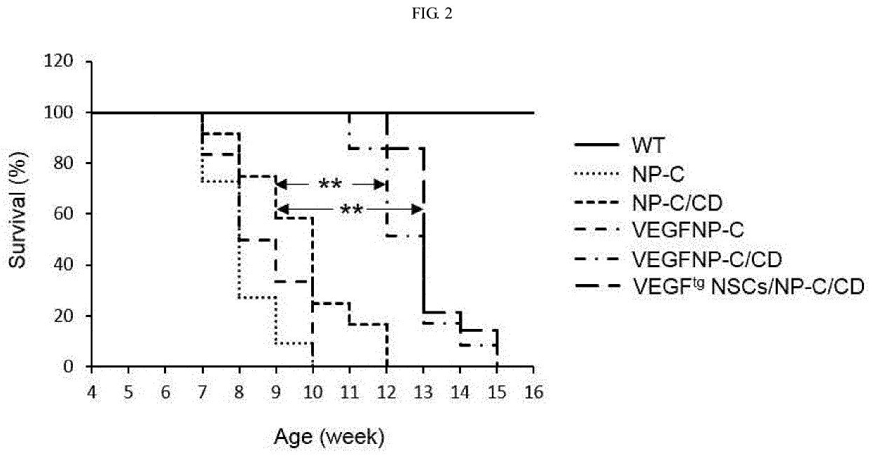

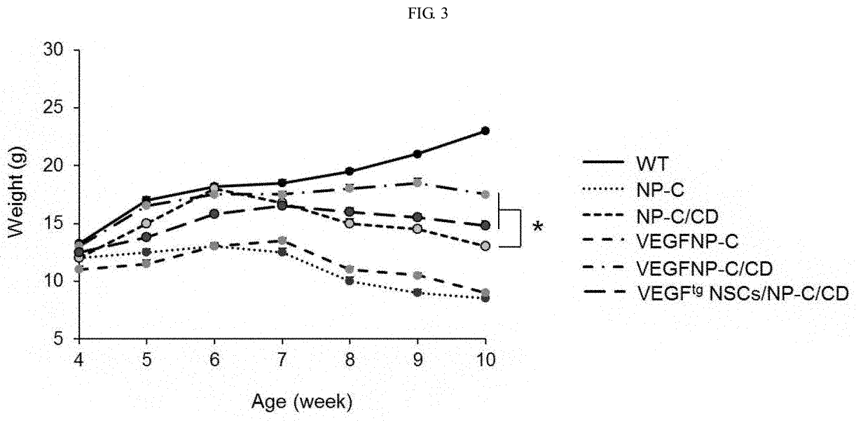

As illustrated in FIGS. 2 and 3, survival rates (FIG. 2) and weights (FIG. 3) of VEGFNP-C mice (VEGFNP-C/CD) injected with cyclodextrin and NP-C mice (VEGFtg NSCs/NP-C/CD) administered with a combination of VEGF-overexpressed neural stem cells and cyclodextrin were significantly increased as compared to NP-C mice (NP-C/CD) administered with cyclodextrin alone (*p<0.05, **p<0.01, n=8-10 per group). In addition, an excellent survival improved effect and a weight loss alleviation effect of the VEGFNP-C/CD group and the VEGFtg NSCs/NP-C/CD group were very excellent compared with the VEGFNP-C group, which showed a somewhat lessened disease state compared to the NP-C mice.

From the above results, it could be seen that the survival rate of NP-C mice was improved and a weight loss was more efficiently alleviated when the brain-specific VEGF was increased and the cyclodextrin was administered in combination, as compared with an effect of administration of cyclodextrin alone or an effect of VEGF alone in neural cells.

### Example 2: Confirmation of Mobility Improvement of NP-C Mice by Brain-Specific VEGF Increase and Administration of Cyclodextrin

In order to confirm whether brain-specific VEGF increase and administration of cyclodextrin improve the decreased mobility of NP-C mice, Rota-rod and Beam tests were conducted for each week age.

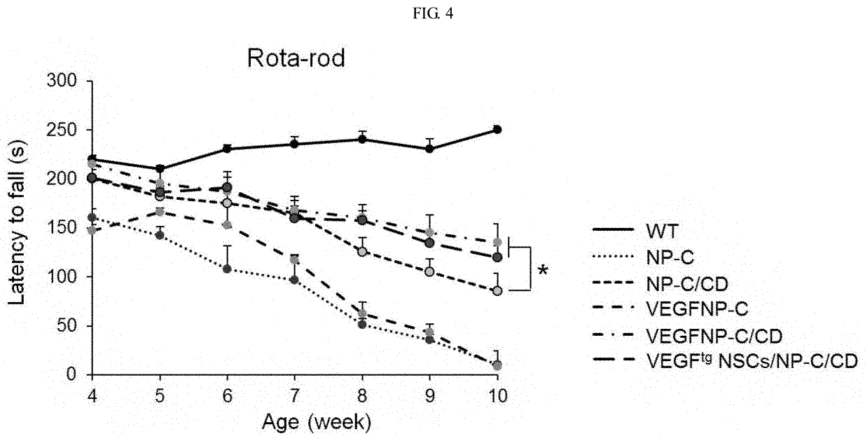

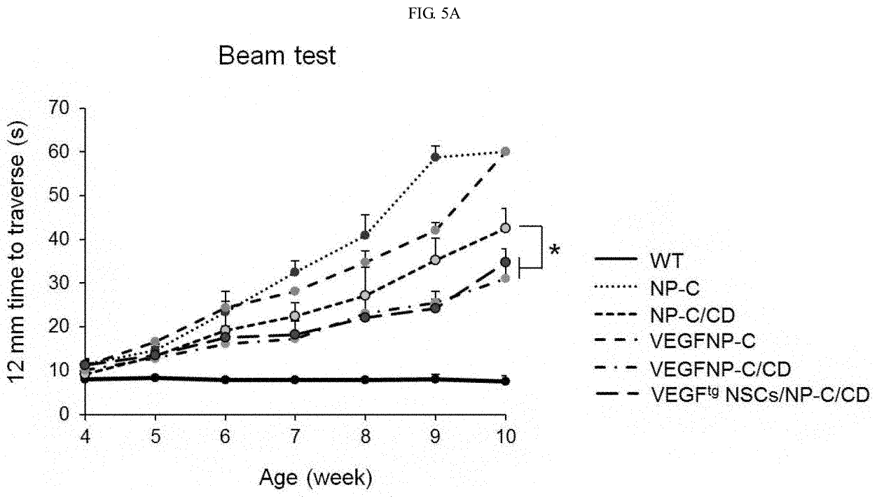

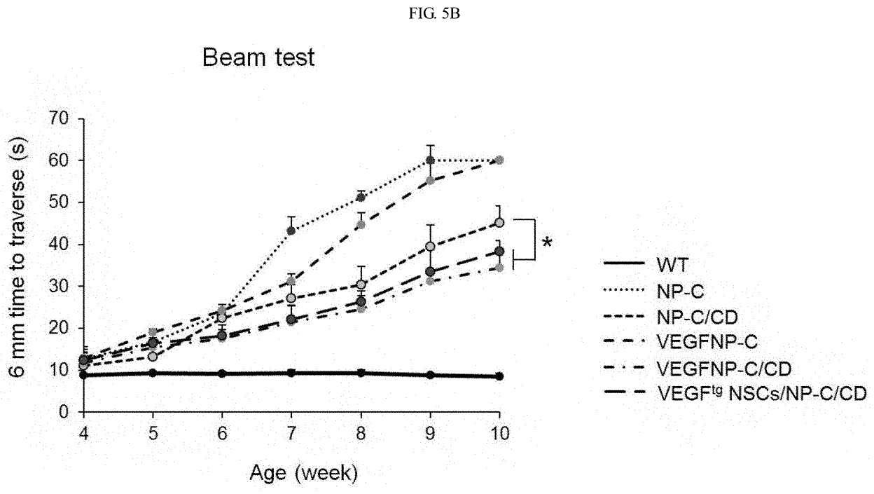

As illustrated in FIGS. 4, 5A, and 5B, as the NP-C mice were aged, the mobility was rapidly reduced, and in the VEGFNP-C mice, the reduction of the mobility was slightly alleviated as compared with the NP-C mice, but as the VEGFNP-C mice wee aged, the mobility was also rapidly reduced. As a result, it was confirmed that the NP-C mice (NP-C/CD) that were administered with cyclodextrin alone had improved mobility compared to NP-C mice, but the improvement effect was most effective in VEGFNP-C mice (VEGFNP-C/CD) injected with cyclodextrin and NP-C mice (VEGFtg NSCs/NP-C/CD) administered with a combination of VEGF-overexpressed neural stem cells and cyclodextrin (*p<0.05, n=8-10 per group). In addition, excellent mobility shown in the VEGFNP-C/CD group and the VEGFtg NSCs/NP-C/CD group was very significant even compared with the VEGFNP-C group, which showed a somewhat alleviated disease state compared to the NP-C mice.

From the above results, it could be seen that the reduced mobility of the NP-C mice was efficiently alleviated when the brain-specific VEGF was increased and the cyclodextrin was administered in combination, as compared with an effect of administration of cyclodextrin alone or an effect of VEGF alone in neural cells.

### Example 3: Confirmation of Alleviation of Cerebral Inflammation of NP-C Mice by Brain-Specific VEGF Increase and Administration of Cyclodextrin

In order to confirm whether brain-specific VEGF increase and administration of cyclodextrin alleviate an increased inflammatory response of the cortex of NP-C mice, the cortex of a mouse of each test group was extracted and subjected to GFAP staining (astrocyte target).

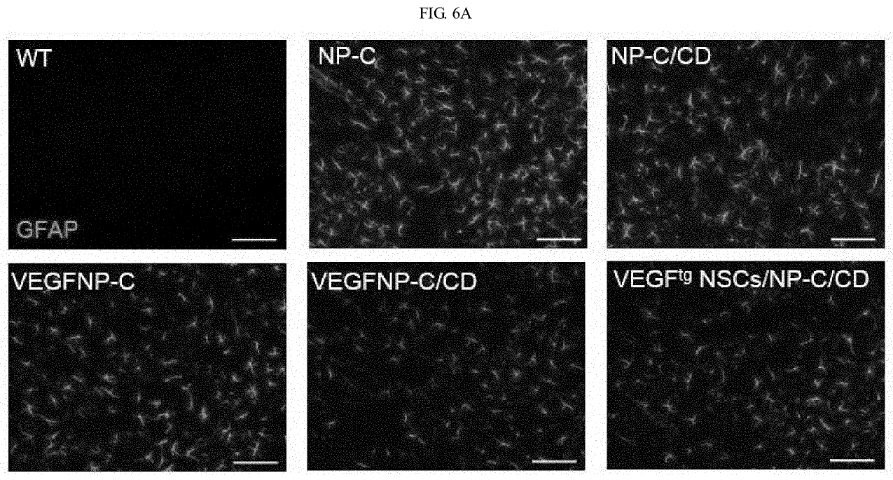

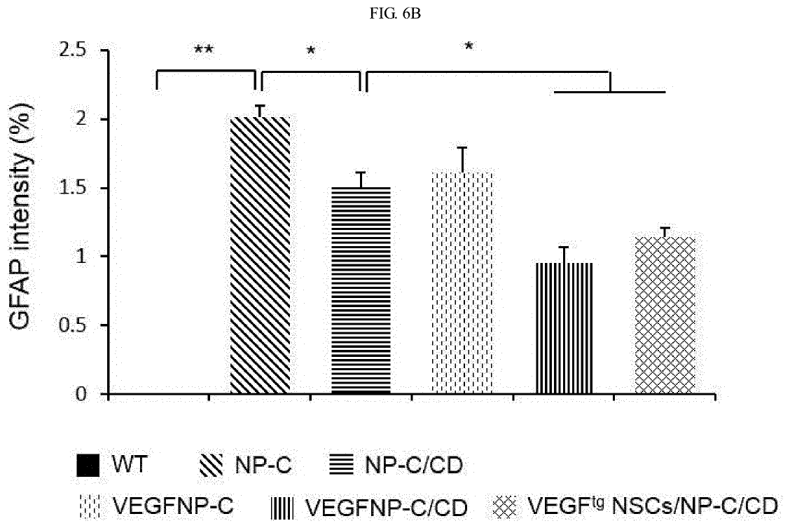

As illustrated in FIGS. 6A and 6B, the increased inflammatory response (GFAP: astrocyte) in the cortex of NP-C mice was somewhat alleviated by administration of cyclodextrin alone (NP-C/CD). In addition, VEGFNP-C mice also showed a cerebral inflammatory response level similar to that of the NP-C/CD group. However, it was confirmed that the inflammation alleviation effect was more effectively improved in VEGFNP-C mice (VEGFNP-C/CD) injected with cyclodextrin and NP-C mice (VEGFtg NSCs/NP-C/CD) administered with a combination of VEGF-overexpressed neural stem cells and cyclodextrin (*p<0.05, **p<0.01, n=3 per group).

From the above results, it could be seen that the cerebral inflammation of the NP-C mice was significantly reduced when the brain-specific VEGF was increased and the cyclodextrin was administered in combination, as compared with an effect of administration of cyclodextrin alone or an effect of VEGF alone in neural cells.

### Example 4: Confirmation of Alleviation of Cerebellar Inflammatory Response and Reduced Cerebellar Neural Cells of NP-C Mice by Brain-Specific VEGF Increase and Administration of Cyclodextrin

In order to confirm whether brain-specific VEGF increase and administration of cyclodextrin alleviate an increased inflammatory response and reduced neural cell symptom of the cerebellum of NP-C mice, the cerebellum of a mouse of each test group was extracted and subjected to GFAP staining (astrocyte target) and Calbindin staining (Purkinje neuron target).

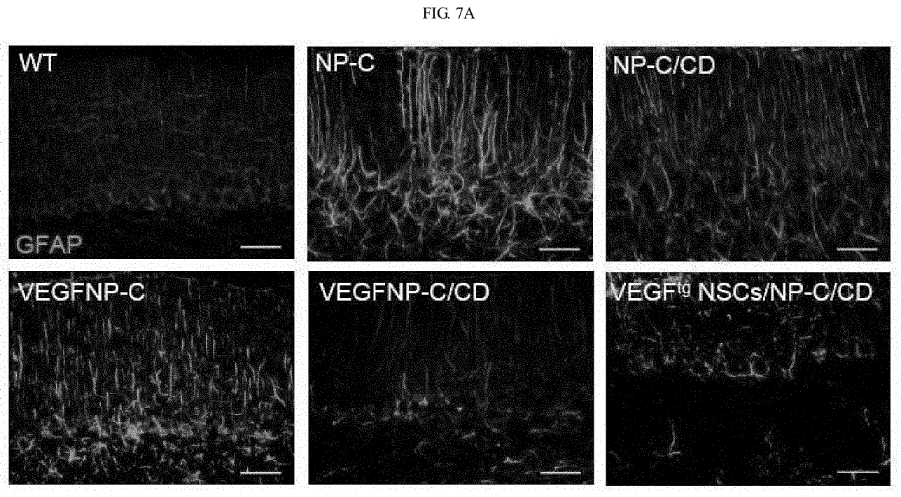

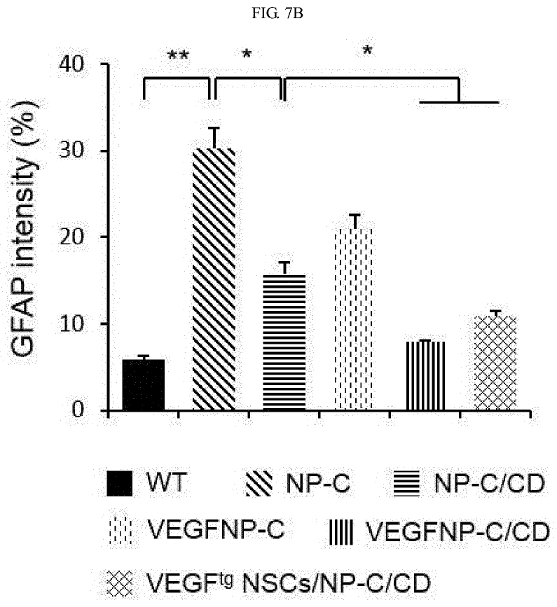

As illustrated in FIGS. 7A and 7B, the increased inflammatory response (GFAP: astrocyte) in the cerebellum of NP-C mice was alleviated to a certain level by administration of cyclodextrin alone (NP-C/CD). However, it was confirmed that the increased inflammatory response was more effectively alleviated in VEGFNP-C mice (VEGFNP-C/CD) injected with cyclodextrin and NP-C mice (VEGFtg NSCs/NP-C/CD) administered with a combination of VEGF-overexpressed neural stem cells and cyclodextrin (*p<0.05, **p<0.01, n=3 per group). In addition, excellent inflammation reduction shown in the VEGFNP-C/CD group and the VEGFtg NSCs/NP-C/CD group was very significant even compared with the VEGFNP-C group, which showed a somewhat alleviated disease state compared to the NP-C mice.

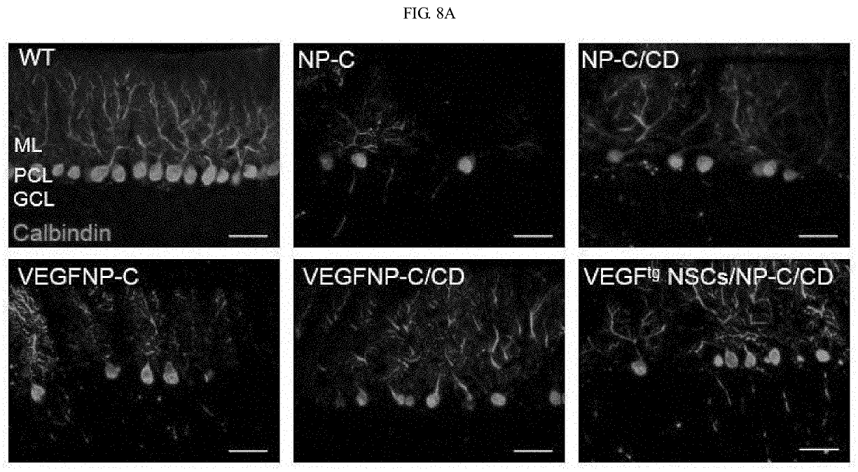

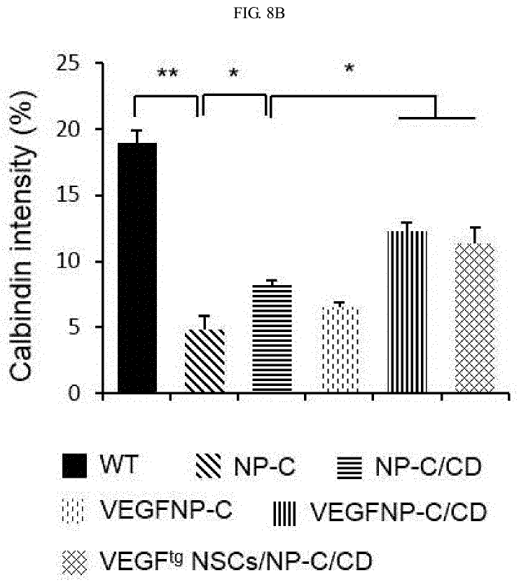

In addition, as illustrated in FIGS. 8A and 8B, it was confirmed that the reduced neural cells (Calbindin: Purkinje neuron) in the cerebellum of the NP-C mice was most efficiently increased in the cerebellum of the VEGFNP-C mice (VEGFNP-C/CD) injected with cyclodextrin and the NP-C mice (VEGFtg NSCs/NP-C/CD) administered with a combination of VEGF-overexpressed neural stem cells and cyclodextrin (*p<0.05, **p<0.01, n=3 per group). Excellent neural cell protection (inhibition of reduced neural cells) shown in the VEGFNP-C/CD group and the VEGFtg NSCs/NP-C/CD group was very significant even compared with the VEGFNP-C group, which showed a somewhat alleviated disease state compared to the NP-C mice.

From the above results, it could be seen that the increase in brain-specific VEGF and the combined administration of cyclodextrin may reduce the increased cerebellar inflammatory response of NP-C mice and alleviate cerebellar neural cell death. In addition, it could be seen that the alleviating effect was more effective than administration of cyclodextrin alone.

From the above results, it could be seen that the cerebellar inflammation of the NP-C mice was significantly reduced when the increase in brain-specific VEGF and administration of the cyclodextrin were combined, as compared with an effect of administration of cyclodextrin alone or an effect of VEGF alone in neural cells.

### Example 5: Confirmation of Reduction of Lipids and Cholesterol Accumulated in Cortex, Cerebellum, and Organs by Increase in Brain-Specific VEGF and Administration of Cyclodextrin

It was confirmed whether an increase in brain-specific VEGF and administration of cyclodextrin affected lipids and cholesterols accumulated in the cortex, the cerebellum and organs of NP-C mice.

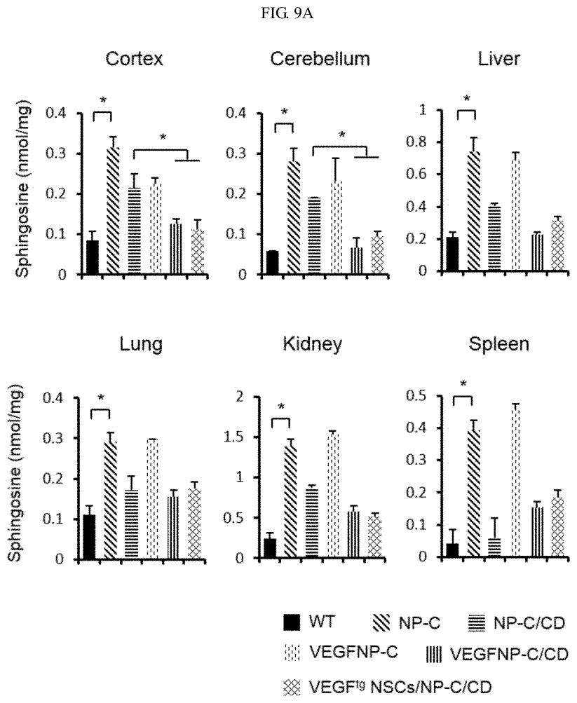

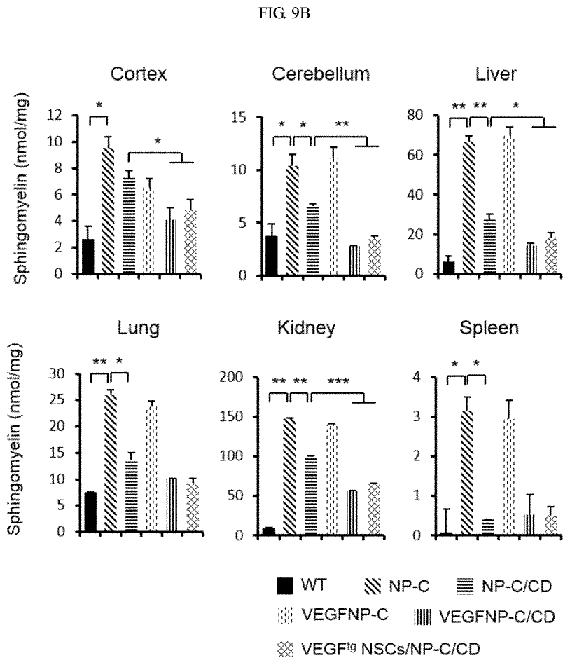

As illustrated in FIGS. 9A and 9B, it was confirmed that sphingosine accumulated in the cortex and the cerebellum of NP-C mice was most effectively reduced in the cortex and the cerebellum of VEGFNP-C mice (VEGFNP-C/CD) injected with cyclodextrin and NP-C mice (VEGFtg NSCs/NP-C/CD) administered with a combination of VEGF-overexpressed neural stem cells and cyclodextrin (FIG. 9A). It was confirmed that in the case of sphingomyelin, the lipid was most effectively reduced even in the liver, the lung, the kidney, and the spleen in addition to the cortex and the cerebellum in the VEGFNP-C/CD group and the VEGFtg NSCs/NP-C/CD group (FIG. 9B) (*p<0.05, **p<0.01, n=3 per group). In addition, excellent lipid reduction shown in the VEGFNP-C/CD group and the VEGFtg NSCs/NP-C/CD group was very significant even compared with the NP-C/CD group and the VEGFNP-C group.

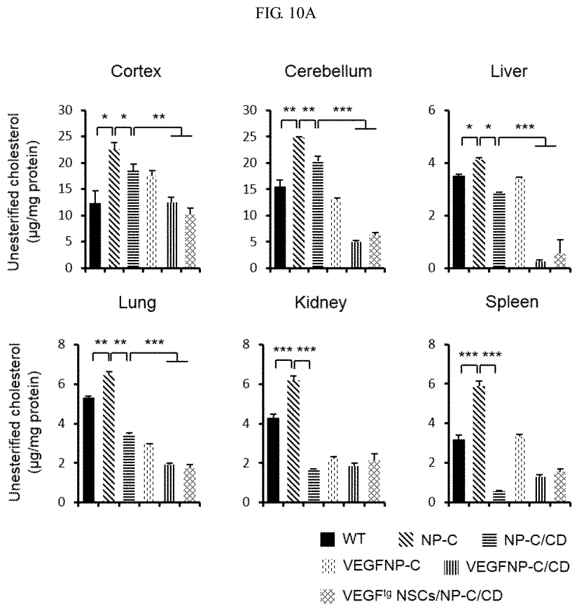

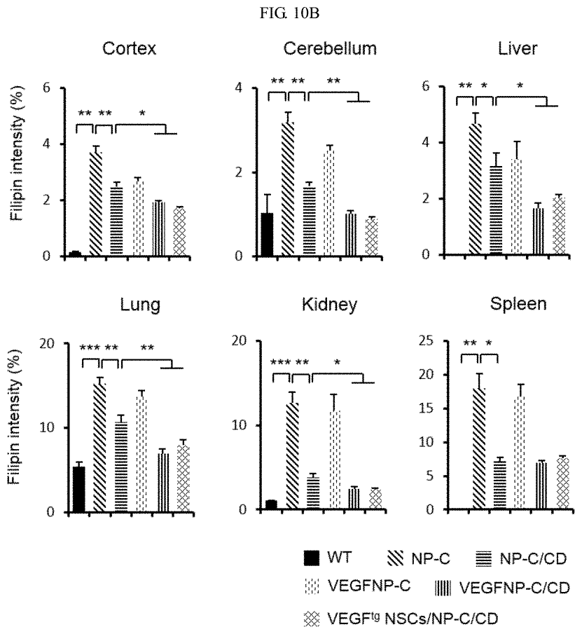

As a result of confirming whether the cholesterol was accumulated, as illustrated in FIGS. 10A and 10B, similarly, it was confirmed that the accumulation of cholesterol was more reduced in the cortex, the cerebellum, and the organs of VEGFNP-C mice (VEGFNP-C/CD) injected with cyclodextrin and NP-C mice (VEGFtg NSCs/NP-C/CD) administered with a combination of VEGF-overexpressed neural stem cells and cyclodextrin (*p<0.05, **p<0.01, n=3 per group).

From this, it could be seen that the effect of reducing accumulation of lipids and cholesterol in the cortex, the cerebellum, and the organs of the NP-C mice was significant when the increase in brain-specific VEGF and the administration of cyclodextrin were combined, as compared with an effect of administration of cyclodextrin alone or an effect of VEGF alone in neural cells.

In summary, it can be seen that if brain-specific VEGF is increased simultaneously with administration of cyclodextrin to the NP-C mice, a lifespan, a weight, mobility, inflammation, neural cell death, and accumulation of lipids and cholesterol in brain and organs of mice may be effectively improved as compared with when cyclodextrin is administrated alone or an increase in brain-specific VEGF is performed alone. From this, it can be seen that the increase in brain-specific VEGF may remarkably enhance a therapeutic effect of cyclodextrin in lipid-related degenerative diseases such as Neiman-pick disease. In addition, it can be seen that the cyclodextrin may remarkably enhance the treatment of the diseases by the increase in brain-specific VEGF.

In other words, in the treatment of lipid-related degenerative diseases such as Neiman-pick disease, the combination of the increase in brain-specific VEGF (particularly, injection of VEGF-overexpressed stem cells) and the administration of cyclodextrin exhibits a synergistic effect on the therapeutic effect of the diseases.

### INDUSTRIAL AVAILABILITY

As described above, the present invention relates to a pharmaceutical composition for preventing or treating neurodegenerative diseases containing, as active ingredients, cyclodextrin and stem cells in which VEGF is overexpressed. A combined treatment of cyclodextrin and stem cells in which VEGF is overexpressed has remarkable synergistic effects, with respect to therapeutic efficacy on the diseases, such as a lifespan increase, mobility improvement, inhibition of neurogenic inflammation, inhibition of neural cell apoptosis and inhibition of lipid accumulation in organs including the brain, in a neurodegenerative disease model, thereby presenting a novel therapeutic strategy. Therefore, there is very high availability in neurodegenerative disease therapeutic agent industry.

1. A pharmaceutical composition for treating neurodegenerative diseases comprising:
cyclodextrin or its pharmaceutically acceptable salt; and
stem cells in which vascular endothelial growth factor (VEGF) is overexpressed, as active ingredients.

2. The pharmaceutical composition of claim 1, wherein the cyclodextrin or its pharmaceutically acceptable salt is administered with a daily dose of 50 mg/day/kg of body weight to 4000 mg/day/kg of body weight.

3. The pharmaceutical composition of claim 1, wherein the stem cells in which the VEGF is overexpressed are administered with a daily dose of 1×10⁵ cells/day to 1×10⁶ cells/day.

4. The pharmaceutical composition of claim 1, wherein the stem cells in which the VEGF is overexpressed are administered simultaneously, separately, or sequentially with the cyclodextrin or its pharmaceutically acceptable salt.

5. The pharmaceutical composition of claim 1, wherein the stem cells in which the VEGF is overexpressed are injected into a subventricular zone (SVZ).

6. The pharmaceutical composition of claim 1, wherein the stem cells are at least one selected from the group consisting of adult stem cells, embryonic stem cells, mesenchymal stem cells, tumor stem cells, and induced pluripotent stem cells.

7. The pharmaceutical composition of claim 6, wherein the adult stem cells are neural stem cells or neural progenitor cells.

8. The pharmaceutical composition of claim 1, wherein the neurodegenerative disease is at least one selected from the group consisting of Neiman-pick disease, Alzheimer's disease, Parkinson's disease, Huntington's disease, Lou Gehrig's disease, schizophrenia, Gaucher disease, Fabry disease, Tay-Sachs disease, Sandhoff disease and cerebellar ataxia.

9. The pharmaceutical composition of claim 8, wherein the Neiman-pick disease is A-type, B-type, C-type, D-type, E-type or F-type Neiman-pick disease.

10. The pharmaceutical composition of claim 1, wherein the composition reduces inflammation of the brain and inhibits accumulation of cholesterol or sphingolipid.

11. A pharmaceutical complex formulation for preventing or treating neurodegenerative diseases comprising the composition of claim 1.

12. The pharmaceutical complex formulation of claim 11, wherein the neurodegenerative disease is at least one selected from the group consisting of Neiman-pick disease, Alzheimer's disease, Parkinson's disease, Huntington's disease, Lou Gehrig's disease, schizophrenia, Gaucher disease, Fabry disease, Tay-Sachs disease, Sandhoff disease and cerebellar ataxia.

13. (canceled)

14. A method for treating neurodegenerative diseases in a subject, the method comprising administering an effective amount of a composition comprising, as active ingredients, cyclodextrin or its pharmaceutically acceptable salt and stem cells in which VEGF is overexpressed to the subject in need thereof.
